(19) United States
(12) Patent Application Publication
(10) Pub. No.: US 2022/0154148 A1
(43) Pub. Date: May 19, 2022

(54) METHODS FOR ENGINEERING HUMAN PLURIPOTENT STEM CELLS FOR DIABETES THERAPY BY CO-TRANSDUCTION

(71) Applicant: Xingxin Wang, Toronto (CA)

(72) Inventor: Xingxin Wang, Toronto (CA)

(21) Appl. No.: 16/951,220

(22) Filed: Nov. 18, 2020

(51) Int. Cl. C12N 5/074

(52) U.S. Cl. CPC C12N 5/0696; C12N 2510/00; C12N 2506/1384

(57) ABSTRACT

Method for preparing and administering human pluripotent stem cells includes preparing human adipose stem cells from a human; epigenetically modifying the human adipose stem cells (hADSC) to yield directly-generated human pluripotent stem cells (dgHPSC); and engineering the dgHPSC to secrete a therapeutic level of insulin. Preparing the human adipose stem cells includes obtaining a lipoaspirate from a human and preparing adipose stem cells from the lipoaspirate. Epigenetically modifying the hADSC includes inducing the hADSC to yield the dgHPSC. Engineering the dgHPSC includes transducing a human estrogen-related receptor gamma (ERRγ) gene into the dgHPSC. Engineering the dgHPSC further includes transducing a human INS gene into the dgHPSC. The dgHPSC transduced with the ERRγ gene and the human INS gene secrete a higher level of insulin compared to the dgHPSC transduced with the ERRγ gene. The engineered dgHPDC are introduced into a human.

### FIELD OF THE INVENTION

The present disclosure relates generally to human pluripotent stem cells. More specifically, the present disclosure describes methods for engineering human pluripotent stem cells for diabetes therapy by co-transduction.

### BACKGROUND OF THE INVENTION

Since the discovery of insulin, daily insulin injection has been generally used for treatment of diabetes with absolute insulin deficiency. However, with the progressing of the symptoms, exogenous insulin administration may need to be intensified with more dosages; and even worse, only administration of insulin cannot maintain blood glucose levels within the narrow physiological range that protects patients from development of various diabetic complications due to insulin injection cannot exactly mimic pancreatic (3 cells to adjust insulin secretion in response to varying blood glucose levels. Currently, available therapies for diabetes have limited effects in preventing the progression of diabetes complications and repairing existing tissue damages. Therefore, improvements of the current diabetes therapies with novel strategies are highly anticipated, to restore the normal functions of pancreatic β cells is critical to finally cure diabetes and protect patients from diabetic complication development.

To generate insulin-producing pancreatic β cells from human embryonic stem cells (hESCs) and human induced pluripotent stem cells (hiPSCs) in vitro provides an unlimited cell source for transplantation therapy in diabetes. So far, a number of groups have generated immature or mature human insulin-producing pancreatic β cells from hESCs and hiPSCs. Although the protocols to generate pancreatic β cell-like cells are complicated and challenging, these achievements are encouraging and promising with the potential for better treatment for human diabetes, further effort in development of the insulin-producing stem cells toward clinic diabetes therapy is highly anticipated.

Estrogen-related receptor γ (ERRγ) is a master regulator of β cell maturation in vivo. Forced expression of ERRγ in hiPSC-derived β-like cells enables glucose-responsive secretion of human insulin in vitro and can further restore the glucose homeostasis in type 1 diabetes mouse models after transplantation, without the need for kidney capsule maturation, to achieve functionality immediately. Human adipose-derived stem cells (hADSCs) were confirmed to have the potentials to differentiate toward the osteogenic, adipogenic, myogenic, chondrogenic, and putative neurogenic cells. Sun et al., reported the successful induction of hiPSCs from hADSCs with lentivirus containing human Oct4, Sox2, Klf4, and c-MYC. However, the use of oncogene c-MYC as one of the inducing factors remains to be a potential concern for clinical application of these hADSC-derived iPSCs.

### BRIEF DESCRIPTION OF THE DRAWINGS

Some of the embodiments will be described in detail, with reference to the following figures, wherein like designations denote like members, wherein:

FIG. 1 illustrates the process step for a method for engineering human pluripotent stem cells for diabetes therapy by co-transduction, according to certain embodiments.

FIG. 2 depicts an inverted phase contrast microscope image of cell morphology of isolated mononuclear cells, according to some embodiments.

FIG. 3 depicts immunofluorescences flow cytometry readings of CD34 and CD44 expression in human adipose stem cells according to other embodiments.

FIG. 4 depicts microscopic images of human adipose stem cell differentiation, according to certain embodiments.

FIG. 5 depicts DTZ staining of directly generated human pluripotent stem cells, according to yet still other embodiments.

FIG. 6 depicts a microscopic image of insulin fluorescent immunochemistry staining of the directly generated human pluripotent stem cells, according to some embodiments.

Unless otherwise specifically noted, articles depicted in the drawings are not necessarily drawn to scale.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

The present disclosure includes many aspects and features. Moreover, while many aspects and features relate to, and are described in the context of method for engineering human pluripotent stem cells for diabetes therapy by co-transduction, embodiments of the present disclosure are not limited to use only in this context.

Since the discovery of insulin, daily insulin injection has been generally used for treatment of diabetes with absolute insulin deficiency. However, with the progressing of the symptoms, exogenous insulin administration may need to be intensified with more dosages; and even worse, only administration of insulin cannot maintain blood glucose levels within the narrow physiological range that protects patients from development of various diabetic complications due to insulin injection cannot exactly mimic pancreatic β cells to adjust insulin secretion in response to varying blood glucose levels. Currently, available therapies for diabetes have limited effects in preventing the progression of diabetes complications and repairing existing tissue damages. Therefore, improvements of the current diabetes therapies with novel strategies are highly anticipated, to restore the normal functions of pancreatic β cells is critical to finally cure diabetes and protect patients from diabetic complication development.

To generate insulin-producing pancreatic β cells from human embryonic stem cells (hESCs) and human induced pluripotent stem cells (hiPSCs) in vitro provides an unlimited cell source for transplantation therapy in diabetes. So far, a number of groups have generated immature or mature human insulin-producing pancreatic β cells from hESCs and hiPSCs. Although the protocols to generate pancreatic β cell-like cells are complicated and challenging, these achievements are encouraging and promising with the potential for better treatment for human diabetes, further effort in development of the insulin-producing stem cells toward clinic diabetes therapy is highly anticipated.

Estrogen-related receptor γ (ERRγ) is a master regulator of β cell maturation in vivo. Forced expression of ERRγ in hiPSC-derived β-like cells enables glucose-responsive secretion of human insulin in vitro and can further restore the glucose homeostasis in type 1 diabetes mouse models after transplantation, without the need for kidney capsule maturation, to achieve functionality immediately. Human adipose-derived stem cells (hADSCs) were confirmed to have the potentials to differentiate toward the osteogenic, adipogenic, myogenic, chondrogenic, and putative neurogenic cells. Sun et al., reported the successful induction of hiPSCs from hADSCs with lentivirus containing human Oct4, Sox2, Klf4, and c-MYC. However, the use of oncogene c-MYC as one of the inducing factors remains to be a potential concern for clinical application of these hADSC-derived iPSCs. This instant disclosure seeks to provide a novel approach, which can directly generate human pluripotent stem cells from hADSCs without any genetic modification, the cells generated by the approach are referred to as “directly-generated human pluripotent stem cells” (dgHPSCs), in addition, to achieve therapeutic level and glucose-responsive insulin secretion, human ERRγ and insulin genes were simultaneously introduced into the dgHPSCs (termed as dgHPSCs+INS+ERRγ) via lentivirus vector transduction.

The instant disclosure seeks to provide a novel concept of developing an insulin therapy for diabetes, centers on a method for developing a human pluripotent stem cell line, dgHPSC, that is induced by a non-genetic modification approach, and the dgHPSC is transduced by lentivirus vector carrying human ERRγ or co-transduced by lentivirus vectors carrying human ERRγ and insulin genes. Expression of human ERRγ gene or co-expression of human ERRγ and insulin genes can synergistically promote the synthesis and secretion of human insulin at the stem cell state, thus the cells do not need to differentiate into matured β-like cells to secret insulin. Transplantation of the transduced dgHPSC cells into diabetic patients may present a promising cell therapy for diabetes.

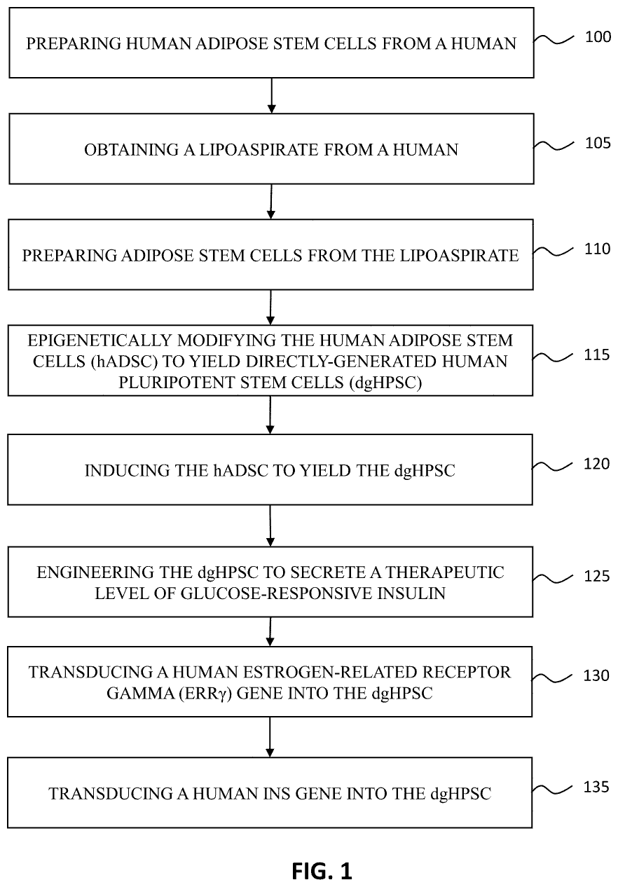

FIG. 1 illustrates the process step for a method for engineering human pluripotent stem cells for diabetes therapy by co-transduction, according to certain embodiments. At Step 100, hADSC are prepared from a human. In preferred embodiments, to achieve Step 100, lipoaspirate is obtained from a human (Step 105) and adipose stem cells are prepared from the lipoaspirate (Step 110). Lipoaspirate was collected by standard abdominal adipose tissue liposuction procedure from volunteered donors. The lipoaspirate tissue was washed using Dulbecco's phosphate buffered saline and centrifuged to remove red blood cells. The resulting pellet was digested at 37° C. for 30 mins with 0.1% collagenase and followed by centrifugation at 800 g for 20 mins to isolate mononuclear cell layer which was a white membrane layer on the top of the liquid in the centrifuge tube.

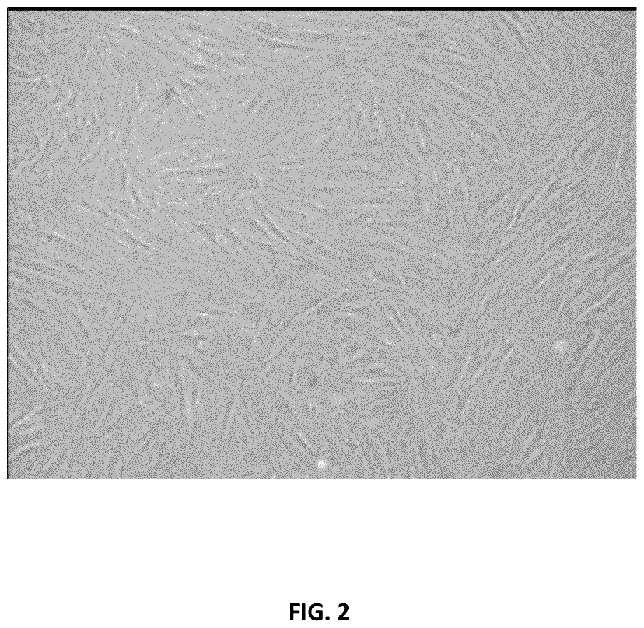

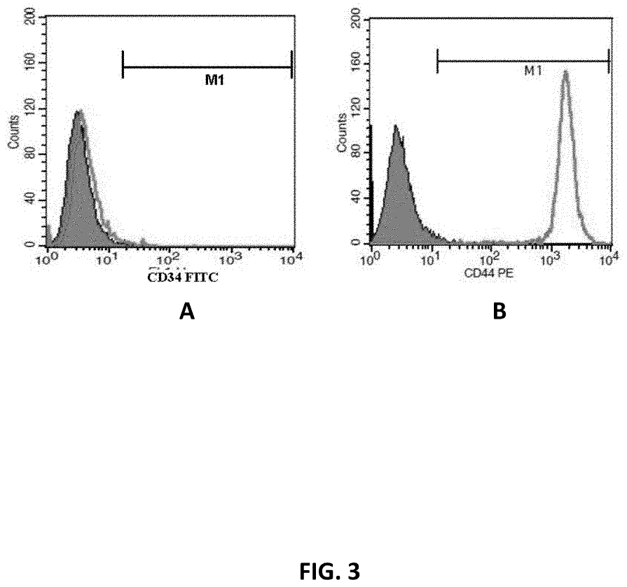

Collagenase digestion was repeated until lipoaspirate tissue was completely digested. Isolated mononuclear cells were cultured using StemPro MSC SFM XenoFree medium with 1% non-essential amino acid, 10 ng/mL SCF and 1% ITS (GIBCO, USA). FIG. 2 depicts an inverted phase contrast microscope image of cell morphology of isolated mononuclear cells, according to some embodiments. Specifically, FIG. 2 depicts cell morphology using an inverted phase contrast microscope. The image shows that the cultured cells were fusiform fibroblast-like and locally swirled. Expression of surface markers of the above fusiform cells was analyzed by immunofluorescence flow cytometry, the cells were labeled with phycoglobin CD44 and FITC CD34 (Biolegend, USA) for flow cytometry by Becton Dickinson FACScan. As reflected in FIG. 3, the results showed that the spindle-like cells expressed CD44, a surface marker of mesenchymal stem cells, but didn't express CD34, a surface marker of hematopoietic stem cells. This stem cell phenotype was consistent with that of adipose stem cells.

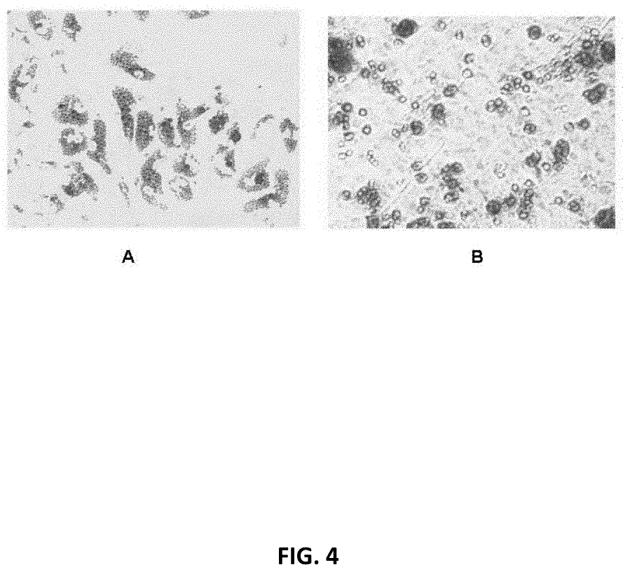

Additional experiments were undertaken to verify the isolation and proliferation of adipose stem cells. First, adipocyte differentiation potential was verified. Here, the passage 3 of morphologically and phenotypically identified cells as described above were transferred to adipogenic induction medium containing base medium DMEM, 10% fetal bovine serum, 0.5 mM IBMX (isobulyl-1-methylxanthione), 0.1 mM indomethacin and 1 μM dexamethasone (Sigma USA). Oil red O staining was performed on day 6, 12 and 16. The results showed vacuoles having an increased refractive index filled in the whole cell at day 12 after incubation with the induction medium. Oil red O staining revealed 60-80% of the cells in the culture were rich in fat droplets (image A of FIG. 4) suggesting that the cells possessed adipocyte differentiation potential in vitro.

Next, osteoblasts differentiation potential in vitro was verified. Here, the passage 3 of the morphologically and phenotypically identified cells were cultured in 6-well plates with osteogenic induction medium which contained basic medium DMEM, 10% fetal bovine serum, 10 mm-glycerin phosphate, 0.1 mM ascorbic acid phosphate Vc, and 0.1 μm dexamethasone. The medium was changed every three days and the cells were cultured for up to 3 weeks. Alizarin red staining was performed at week 2 and week 3 respectively. The results showed that the cells in the osteogenic induction culture were transformed from the spindle-like shape to cubic-like and formed a multi-layer nodule structure. Two weeks after induction, mineralized nodules were formed; and after 3 weeks, more nodules and intense Alizarin red stain could be observed (image B of FIG. 4). This result suggests that the adipose stem cells possessed osteoblasts differentiation potential in vitro.

At Step 115, the hADSC are epigenetically modified to yield dgHPSC. In preferred embodiments, Step 115 is accomplished by inducing the hADSC to yield the dgHPSC (Step 120). An induction medium was used to induce dgHPSCs without the need of gene modification. TRA-1-60 live staining was used for identifying successfully reprogrammed colonies, based on previous report that along with assessing morphological differences, staining with TRA-1-60—specific antibody can be used to distinguish successfully reprogrammed colonies from other transformed non-iPSC colonies. The cells were cultured with the induction medium for 14 days and TRA-1-60 immuno-fluorescent stain was performed. Briefly, the cells in 24-well cell culture plate were incubated with diluted mouse IgM TRA-1-60 (Millipore, USA) and Alexa Fluor 555—conjugated secondary anti-mouse IgM antibody (Invitrogen, USA) in the induction medium for 1 hour, washed twice with PBS and replaced with 1 ml induction medium and examined under fluorescent microscope.

At Step 125, the dgHPSC are engineered to secrete a therapeutic level of insulin. In preferred embodiments, Step 125 includes transducing ERRγ gene into the dgHPSC (Step 130) and/or transducing the human INS (insulin) gene into the dgHPSC (Step 135).

Here, use of a lentivirus vector is preferred. Insulin lentivirus gene vector pWPI/INS and ERRγ lentivirus gene vector pWPI/ERRγ were constructed using original vector pWPI/hPLKWT/Neo (Addgene plasmid #35385). Briefly, the DNAs of pWPI/hPLKWT/Neo vector and insert genes, human insulin and ERRγ, were digested with BamH I restriction enzyme (New England Biolabs, USA), separated by 1% Agarose Gel Electrophoresis, and recovered by QIAGEN Gel Extraction kit (QIAGEN, Germany). The recovered pWPI vector and inserts were ligated with T4 DNA ligase (New England BioLabs) respectively and transformed into Top 10 competent cells according to the manufacturer's instructions (Invitrogen, USA). The positive colonies were primarily analyzed by BamH I digestion and further confirmed by DNA sequencing.

The pWPI/INS lentiviruses or pWPI/ERRγ lentiviruses were produced in HEK293T cells by transfection of pWPI/INS or pWPI/ERRγ lentivirus vectors, respectively, with packaging vectors psPAX2 (Addgene plasmid #12260) and pMD2.G (Addgene plasmid #12259) using Lipofectamine 2000. Briefly, HEK293T cells were cultured until 90% confluence, the transfer vectors, pWPI/INS or pWPI/ERRγ with the packaging vectors, psPAX2 and pMD2.G, respectively, were co-transfected by Lipofectamine 2000 according to the manufacturer's instructions. The cells were incubated at 37° C., 5% CO₂ incubator for at least 6 hours or overnight. Transfection reagents were removed and the cells incubated with normal culture medium for 48 hours after transfection. The supernatant was collected and centrifuged at 2000 g for 20 minutes and then filtrated via 0.45 μm filters.

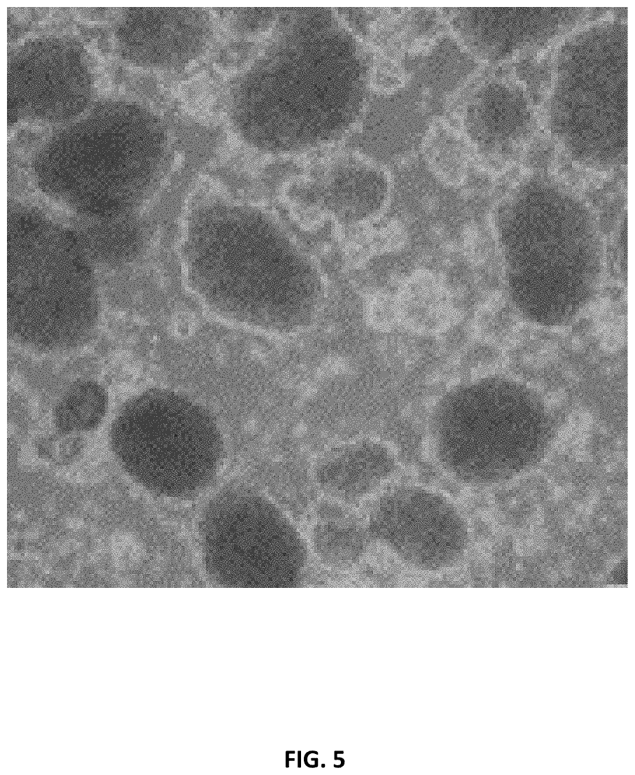

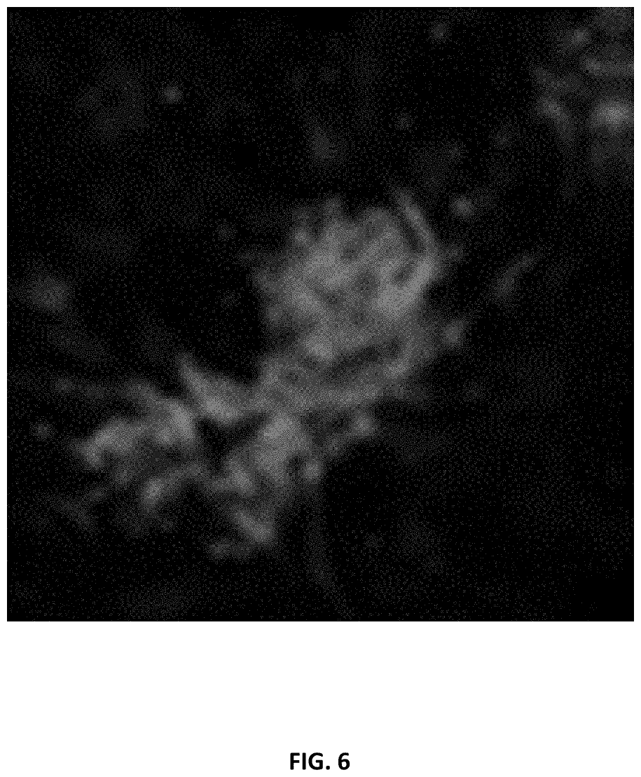

The dgHPSCs were cultured in 15-cm dishes with DMEM (Dulbecco's Modified Eagle Medium) and 10% fetal bovine serum. After removal of cell culture medium, pWPI/INS lentiviruses or pWPI/ERRγ lentiviruses or a mix of pWPI/INS and pWPI/ERRγ lentiviruses in 25 ml filtrated cell culture medium of the transfected HEK293T cells were added into each dish and incubated for 6 hours to overnight to transduce the dgHPSCs. Insulin secretion of dgHPSCs was detected 24 hours after infection with pWPI/INS or pWPI/ERRγ lentiviruses or co-infection with mix of pWPI/INS and pWPI/ERRγ lentiviruses. Insulin secretion peaked 72 h after infection as revealed by DTZ (diphenylthiocarbazone) brownish red stain (FIG. 5) and insulin fluorescent immunochemistry stain (FIG. 6).

The dgHPSCs cells were infected with pWPI/ERRγ lentiviruses or pWPI/INS lentiviruses, or both pWPI/ERRγ and pWPI/INS lentiviruses. Two days post infection, insulin in the cell culture supernatants was tested. The concentration of insulin in the supernatant of pWPI/ERRγ lentiviruses infected dgHPSCs (dgHPSCs+ERRγ) was 30.84 μIU/ml, whereas that of pWPI/INS lentiviruses infected dgHPSCs (dgHPSCs+INS) was 11.61 μIU/ml and that of pWPI/ERRγ plus pWPI/INS lentiviruses infected dgHPSCs (dgHPSCs+ERRγ+INS) was 84.47 μIU/ml which was much higher than that of the dgHPSCs+INS cells or dgHPSCs+ERRγ cells.

Therefore, human pluripotent stem cells overexpressing ERRγ can efficiently synthesize and secrete human insulin at the pluripotent stem cell state, do not need to differentiate into β-like cells; and in addition, co-expression of human insulin and ERRγ genes can synergistically further promote synthesis and secretion of human insulin in dgHPSC cells. Insulin quantities secreted into cell culture supernatant was tested by an electrochemiluminescence method performed by Kingmed Diagnostics (Jinan, China). Human pluripotent stem cells overexpressing ERRγ or co-expression of human insulin and ERRγ are preferably introduced into humans.

Although the disclosure has been explained in relation to its preferred embodiment, it is to be understood that many other possible modifications and variations can be made without departing from the spirit and scope of the disclosure.

What is claimed is:

1. Method for preparing and administering human pluripotent stem cells, comprising:
preparing human adipose stem cells from a human;
epigenetically modifying the human adipose stem cells (hADSC) to yield directly-generated human pluripotent stem cells (dgHPSC); and
engineering the dgHPSC to secrete a therapeutic level of insulin.

2. The method of claim 1, wherein
the step of preparing the human adipose stem cells comprises:
obtaining a lipoaspirate from a human; and
preparing adipose stem cells from the lipoaspirate.

3. The method of claim 2, wherein
epigenetically modifying the hADSC comprises inducing the hADSC to yield the dgHPSC.

4. The method of claim 3, wherein
engineering the dgHPSC comprises transducing a human estrogen-related receptor gamma (ERRγ) gene into the dgHPSC.

5. The method of claim 4, wherein
engineering the dgHPSC further comprises transducing a human INS gene into the dgHPSC; and
the dgHPSC transduced with the ERRγ gene and the human INS gene secrete a higher level of insulin compared to the dgHPSC transduced with the ERRγ gene.

6. The method of claim 5, further comprising:
introducing the engineered dgHPDC into a human.

7. Method for preparing and administering human pluripotent stem cells, comprising:
preparing human adipose stem cells from a human by:
obtaining a lipoaspirate from a human;
preparing adipose stem cells from the lipoaspirate;
epigenetically modifying the human adipose stem cells (hADSC) to yield directly-generated human pluripotent stem cells (dgHPSC); and
engineering the dgHPSC to secrete a therapeutic level of insulin.

8. The method of claim 7, wherein
epigenetically modifying the hADSC comprises inducing the hADSC to yield the dgHPSC.

9. The method of claim 8, wherein
engineering the dgHPSC comprises transducing a human estrogen-related receptor (ERR) gamma gene into the dgHPSC;

10. The method of claim 9, wherein
engineering the dgHPSC further comprises transducing a human INS gene into the dgHPSC; and
the dgHPSC transduced with the ERRgamma gene and the human INS gene secrete a higher level of insulin compared to the dgHPSC transduced with the ERRgamma gene.

11. The method of claim 10, further comprising:
introducing the engineered dgHPSC into a human.

12. Method for preparing and administering human pluripotent stem cells, comprising:
preparing human adipose stem cells from a human;
epigenetically modifying the human adipose stem cells (hADSC) to yield directly generated human pluripotent stem cells (dgHPSC);
engineering the dgHPSC to secrete a therapeutic level of insulin;
wherein epigenetically modifying the hADSC comprises transducing a human estrogen-related receptor (ERR) gamma gene and a human INS gene into the dgHPSC; and
wherein the dgHPSC transduced with the ERRgamma gene and the human INS gene secrete a higher level of insulin compared to the dgHPSC transduced with the ERRgamma gene

13. The method of claim 12, wherein
the step of preparing the human adipose stem cells comprises:
obtaining a lipoaspirate from a human; and
preparing adipose stem cells from the lipoaspirate.

14. The method of claim 13, wherein
epigenetically modifying the hADSC comprises inducing the hADSC to yield the dgHPSC.

15. The method of claim 14, further comprising:
introducing the engineered dgHPSC into a human.
